(19) United States
(12) Patent Application Publication
(10) Pub. No.: US 2022/0165423 A1
(43) Pub. Date: May 26, 2022

(54) SYSTEMS AND METHODS FOR DIAGNOSTIC TESTING

(71) Applicant: Binx Health Limited, Trowbridge (GB)

(72) Inventors: Jeffrey Luber, Cambridge, MA (US); Marc Jones, Trowbridge (GB); Alex Kramer, Trowbridge (GB)

(21) Appl. No.: 17/533,969

(22) Filed: Nov. 23, 2021

Related U.S. Application Data
(60) Provisional application No. 63/118,406, filed on Nov. 25, 2020.

(51) Int. Cl. G16H 50/20; G16H 10/60

(52) U.S. Cl. CPC G16H 50/20; G16H 10/60

(57) ABSTRACT

The invention provides a system for the contact-limited or contact-free diagnostic testing. The system offers convenient app or web-based algorithms for obtaining diagnostic care without the need for consulting a physician prior to requesting the test. Specifically, the system provides a user interface portal for an individual to interact with testing laboratories in order to request diagnostic testing, receive physician approval, receive an in-home test kit, and for anonymized results returned to the requesting individual through the app or web-based interface. By providing a patient management portal system useful for initiating and managing interactions between individuals and the testing laboratories, the invention bridges point-of-care with at-home care thus improving access to potentially life-saving diagnostic testing.

### TECHNICAL FIELD

The invention relates to systems for contact-free diagnostic testing of individuals.

### BACKGROUND

One problem that arises in health care is a disconnect between people's regular doctor visits and people's exposure to medical threats. Even people who visit their primary care physician annually for a check-up may be exposed to infectious diseases unexpectedly at any time. People may be worried that they've been exposed to a virus or other contagion but may not find any appointments immediately available at their local doctor's office. Needless to say, for many types of contagious diseases, rapid detection and treatment is essential to not only the health of the individual but also to stopping the spread in a community.

Rapid disease detection faces both social and structural obstacles. For example, certain categories of infections, such as sexually transmitted infections (STIs) may carry social stigma that understandably make people reluctant to seek a test. A structural obstacle to rapid disease detection lies in emerging patterns of national and global health care access and engagement. For example, in the United States it is estimated that as many as 25% of people do not even have a primary care provider. That number is understood to be larger in other parts of the world and growing among vulnerable segments of the population. Due to the lack of reliable access to regular, primary medical care, some people are exposed to infectious diseases without good opportunities to promptly consult with a doctor. Those barriers to access result in real and measurable harms to individuals exposed to diseases. Worse yet, those barriers to access allow infectious diseases to spread unchecked through populations.

### SUMMARY

The invention provides systems and methods useful for testing people for diseases without requiring people to see a doctor before the test is performed. A person concerned about disease exposure may request a test a through an online system of the invention. The system collects certain clinically-relevant information from the person and coordinates with a clinical services laboratory and a medical provider or physician to have the physician approve (or “order”) the test and also to have a test kit shipped to the person. From the person's point-of-view, he or she inquires about a test through a patient portal, e.g., provided as a smartphone app or webpage. After answering any qualifying questions, the person receives a test kit, e.g., in the mail. Behind the scenes, the system has presented qualifying information to the physician, received authorization for the test, coordinated with the clinical services laboratory for shipment of the test kit, and managed the digital records that will allow for authentication or activation of the test kit and management and delivery of laboratory results.

The person uses the test kit in the privacy of their own home to collect and send a sample (e.g., using a swab, tube, or finger-prick) to the clinical services laboratory. The clinical services laboratory performs an assay (e.g., a polymerase chain reaction test for a viral genetic sequence, an enzyme-linked immunosorbent assay for a protein, etc.) and uploads results to the system. With any required physician authorization, a test result (e.g., positive or negative for infection) may be provided to the person back through the same patient portal, e.g., smartphone app or webpage. The system may be implemented in a server or cloud environment with protections to ensure appropriate confidentiality, sample chain-of-custody, and regulatory compliance. For example, in embodiments, the clinical services laboratory ships a test kit with a barcode or QR code on the seal and the system only opens a record for test results once the person counter-activates the test by uploading a picture from the code. Because the system manages back-end coordination with the medical provider (e.g., physician), any of the sample collection, laboratory assay, and test result may be provided without requiring the patient to have met the medical provider face-to-face.

In fact, the system is useful for “bulk approvals” by which the medical provider authorizes, or orders, multiple tests for multiple individuals by one action, e.g., one mouse-click. For example, an institution such as a college may wish to screen an entire cohort of people for one medical condition at the same time. The institution may instruct the members to register and request a test. The system can collect qualifying information and distill and aggregate relevant information. The system can then, present an aggregate test request to a medical provider who can authorize or order multiples of the test en masse. The order for multiple tests can be sent to a clinical services laboratory to coordinate delivery of multiple test kits, one to each member of the cohort. In such a situation, each member can open his or her kit (optionally with authentication or activation), perform sample collection (e.g., swab, spit, or skin-prick), and send the sample to the clinical services laboratory. The laboratory can perform the indicated assay on any or all of the received samples and upload results through the system after which the system can coordinate physician review of, and member access to, test results.

Those systems and methods of the invention are useful for detection disease promptly when people may have been exposed to a disease. People may order tests without scheduling a visit with a doctor. Removing the initial requirement for a doctor visit lowers barriers associated with social stigma or structural disengagement with the health care system. As such, system and methods of the invention promote rapid disease detection and early, and thus effective, treatment, giving individuals optimal outcomes after disease exposure. Moreover, bulk (or aggregate) test approvals and management give institutions and populations important tools for stopping and preventing the spread of infectious diseases.

Systems of the invention offer convenient app or web-based algorithms for obtaining diagnostic care without the need for consulting a physician prior to requesting the test. Specifically, the system provides a patient portal for an individual to interact with testing laboratories in order to request diagnostic testing, receive physician approval, receive an in-home test kit, and for anonymized results returned to the requesting individual through the app or web-based interface.

The patient portal may exist on the user end as a dashboard through which an individual may sign-up and request an at-home diagnostic test. The request may be transmitted through the interface to the clinical testing laboratory where the clinical testing laboratory interacts directly with the individual and facilitates the ordering and approval of the test. The clinical testing laboratory may ship (or order shipping of) the test to the individual, confidentially and securely activate the test, and provide results to the individual in an anonymized fashion via the patient portal. All aspects of the interface are preferably managed through the system by the clinical testing lab thus streamlining access to diagnostic care. Accordingly, the invention is useful for initiating and managing interactions between individuals and the testing laboratories, and bridges point-of-care with at-home care thus improving access to potentially life-saving diagnostic testing.

Aspects of the invention provide a testing method. The method includes obtaining, by a computer system, a request from a person for a medical test and answers to questions selected based on the medical test; sharing the request and answers with a medical provider; and recording from the medical provider an order for the medical test. The computer system is further operated for authorizing shipment of a medical test kit to the person; accepting input of a test result from the laboratory; and providing the person with access to the test result. The obtaining step may include having the person sign-in to access the system, make the request for the medical test, and give the answers to the questions selected based on the medical test. The system may present the person with the questions in response to receiving the request for the medical test from the person. Optionally, the authorizing step includes informing the laboratory that the medical provider ordered the test and notifying the person that the test kit is activated.

The computer system (e.g., a server and/or cloud-based hardware system that includes at least one processor coupled to non-transitory memory containing instructions useful to cause the computer system to perform the method) may be operated to: provide a patient portal to each of a plurality of test subjects, each via a display in a browser or app; provide a physician portal to at least one medical provider; and operate, for the laboratory, a laboratory dashboard allowing laboratory personnel to manage test requests and results.

In some embodiments, the person is shown the questions selected based on the medical test through his or her respective patient portal. The computer system may provide functionality allowing the laboratory to activate the medical test kit. Preferably, the computer system via a patient portal confirms receipt by the person of the activated medical test kit. Optionally, the received request is for a medical test for an infection, and the order requires the medical test kit to include a collection device for a biological sample, and the test result input by the laboratory is obtained by performing a laboratory assay on the biological sample.

In certain embodiments, the infection is a sexually transmitted infection. The collection device includes a genital swab and the laboratory assay may include a test for the presence of one or more molecular markers of, for example, chlamydia and/or gonorrhea. In other embodiments, the infection is a virus such as a respiratory virus. The collection device includes a nasal swab and the laboratory assay includes a test for the presence of one or more molecular markers of the respiratory infection.

In en masse embodiments, the computer system receives requests from a plurality of people for the same test. The computer system: aggregates test requests based on clinical similarities in a plurality of answers from the respective people; shares a single aggregate request with the medical provider; and records from the medical provider a bulk authorization of orders for medical tests for the plurality of people.

The computer system may be operable to: operate, for the laboratory, a laboratory dashboard allowing laboratory personnel to manage test requests and results; and read from, and update, records in a laboratory information management system (LIMS) of the laboratory. Optionally, the computer system is operable to: provide a physician portal to at least one medical provider and authorize the laboratory to ship the medical test after (and only after) recording from the medical provider the order for the medical test. The step of providing the person with access to the test result may be performed before the person has met the medical provider face-to-face. The test result may include the presence or absence of an infection such as a sexually-transmitted infection.

The computer system may aggregate test requests from a plurality of people into a single aggregate request to the medical provider; and authorize, upon receiving an approval of the aggregate request from the medical provider, the laboratory to ship test kits to each of the plurality of people. The computer system may operate a laboratory dashboard to receive and manage results from the test kits, in which the results include test results for an epidemic or pandemic disease or condition.

In certain embodiments, the computer system includes a machine learning system that operates on the answers to make a patient classification or test recommendation. The patient classification or the test recommendation may be provided to the laboratory or the medical provider.

The computer system may be operable to: provide a physician portal to the medical provider; screen the request based on clinical guidelines for the medical test; and present the screened request to the medical provider via the physician portal. In some embodiments, authorization for the requested test triggers distribution of the test kit to the requesting individual. The method may include providing the medical test kit to the person by sending to the medical test kit to: an address for the person, or a participating pharmacy or clinical site. The authorizing step may include informing the laboratory that the medical provider ordered the test and prompting the person to activate the test. The computer system may document a chain of custody for the medical test kit and results.

In some aspects, the invention provides a contact-free system or method for diagnostic testing in an individual. The method comprises providing the requesting individual a user interface portal wherein the user interface is a computer display providing the individual a set of questions specific to the test requested; providing a user input for receiving the individual's answers to the set of questions; transmitting to a clinical testing laboratory the individual's answers to the set of questions specific to the requested test; transmitting, through the system, the individual's answers to the set of questions to a medical provider's dashboard for approval for the requested test; receiving the medical provider's authorization for distributing the test to the individual; providing to the individual the medical provider's authorization for the requested test; providing to the individual the requested test kit; activating the individual's test kit; providing instructions and a means for returning the kit to the clinical testing laboratory for testing; testing the sample for the requested test; and transmitting results back to the individual through the user interface.

The system provides an algorithm for the interface between the requesting individual, the clinical testing laboratory, the medical provider approval, sending and receiving the test kit, analyzing the sample, and returning results to the patient. In preferred embodiments, the user interface is a computer with or without a touch screen. Relatedly, the system provides a user dashboard portal wherein the individual may interact with the system to sign in, request a test, and answer questions specific to the requested test. Ideally, the user interface is an app or is web-based. In the system, the user dashboard portal interfaces with the diagnostic clinical testing laboratory, which also has a dashboard. Also within the system, the clinical testing laboratory dashboard interfaces with a dashboard provided for the medical provider approving test requests.

The invention contemplates the user dashboard portal display provides the individual a set of questions specific to the diagnostic test requested, such as questions related to a suspected infectious or communicable disease or health condition of interest. The user interface comprises an input for receiving the requesting individual's answer to the set of questions. The individual desiring a diagnostic test may also enter demographic information such as age, gender, or zip code. In some embodiments, the user dashboard includes applications in and translation to various languages to allow for access by non-English-speaking populations.

Systems and methods of the invention provides a means for medical provider approval of the requested test. Answers to the specific questions are received by the clinical testing laboratory which then transmits the information to the medical provider for approval. The request for approval is received on the medical provider dashboard of the system. The request is screened based on clinical guidelines for approval of the test. In one aspect of the invention, the input data such as answers to questions, are fed into specific machine learning models/algorithms such as a recommendation system, classification system, or ensemble algorithm which has been trained on multiple separate data sets. The set of questions may be a set compliant with FDA regulations for test issuance. Approval may also be conducted through a machine learning algorithm developed through codification of current clinical algorithms and further trained on multiple separate data sets.

Once the medical provider approves test kit delivery to the requesting individual, approval is transmitted through the system to the clinical testing laboratory dashboard. Proof of the medical provider's authorization may be provided to the individual on a printed paper, or electronically, by email, through two-factor authentication, through a proprietary application, or as a scannable barcode, for example a quick response (QR) code. In some embodiments, proof of the medical provider's authorization may be sent directly from the clinical testing laboratory dashboard to a pharmacist, with the individual optionally receiving a copy of the authorization.

The system provides that authorization received within the clinical testing laboratory dashboard interface triggers the clinical testing laboratory to distribute the test kit to the patient for testing. Providing the requesting individual the test kit may comprise sending the kit to the individual's address through mail or other delivery service, or distributing to a kiosk, pharmacy, or other predetermined secure location. The samples may have end-to-end chain of custody provided, tracked, or verified by a system of the invention. The clinical testing lab ensures the location of the test kit is at all times tracked. This may be achieved through scanned bar codes, or bulk shipping for test distribution. Mobile vans may be used to retrieve samples such as outside a dorm, in a neighborhood at UPS, etc.

Methods of the invention may include activating the individual's test kit. In some embodiments, the user dashboard portal provides instructions to the individual for activating the test kit. Once received, the user interface portal provides instructions through the user dashboard for activating the test kit. In related aspects, activation may contain security features such as electronic patient verification or biometric verification, or bar code or QR code to ensure patient confidentiality and verification. The system also provides instructions for completing and returning the test kit for analysis and processing. In some embodiments, instructions are provided through the user dashboard portal. The packaging for the test may comprise a completed and/or pre-paid shipping label as well as appropriate biohazard labeling and packing material. In related aspects, the return packaging may include tracking information relayed through the system to the clinical testing laboratory dashboard and the user dashboard.

The returned test kit is then analyzed by the clinical testing lab and the results of the processed tests are provided to the requesting individual. Providing the results to the individual may comprise electronically transmitting the results to the individual through the user dashboard, or by email or physical means such as mail, shipping, or courier.

The invention provides for embodiments wherein an individual may request a test kit for any number of tests that can be performed with for example blood, saliva, pus, excrement, urine, or other bodily fluid. Common tests include HbA1c, cholesterol and lipids test, Fit Colon Cancer Screening, HPV, metabolism, vitamin D and testosterone, vitamin D and inflammation, and sexually transmitted infections. Other examples include tests for iron deficiency or high cholesterol. Tests requested could also be for an enzyme marker such as alkaline phosphatase (ALP), alanine aminotransferase (ALT), aspartate aminotransferase (AST), or bilirubin for risk of cancer or other conditions such as liver cirrhosis, gallbladder inflammation, stroke, hepatitis, or celiac disease. The requested test kit could be for thyroid function panel, kidney function test, or glucose test. An individual may also request various coagulation tests such as a Factor V, Fibrinogen level, prothrombin time, or platelet count. Further the test requested could be for hormone levels indicative of health conditions such as a dehydroepiandrosterone (DHEA) deficiency in men which may indicate type 2 diabetes, kidney disease, or AIDS, where high levels in men or women may indicate cancer or tumor in adrenal glands. Other tests requested may be protein-related tests such as C-reactive protein test (CRP) high levels of which may indicate artery inflammation, inflammatory bowel disease, lupus, rheumatoid arthritis, or heart disease. Other screening tests may include screenings for colorectal cancer or allergic or food sensitivities.

In other embodiments, testing may also be for pathogenic diseases such as HIV, hepatitis C, COVID-19, or for common bacteriological infections such as strep. The self-administered at-home test kit for pathogen testing provides the advantage of avoiding interaction between the person and a medical worker who would otherwise be required to administer the test or collect a sample, preventing an opportunity for the pathogen to spread to the medical worker.

In another aspect, the invention provides for mass diagnostic testing. The clinical testing laboratory may receive identical or related requests from multiple individuals for diagnostic testing. For example, if a university requires students to be tested for COVID-19 before they are allowed on campus, the university may have students sign up through the system with their own user dashboard and request a COVID-19 test. As above, the student will enter demographic data and answer questions related to the test kit requested. The requests are received from individuals via the clinical testing laboratory dashboard and are presented to clinicians via the medical provider's dashboard for approval en masse. Relatedly, the medical provider's dashboard is code-compliant for mass approvals. Once approval is received by the clinical testing laboratory, appropriate test kits are sent to the requesting individuals with instructions for use. The completed kits are then sent back to the clinical laboratory for analysis and processing. Results are reported on an individual basis to each requesting individual through their specific user dashboard.

In related aspects, the test kit requests may come from a care-provider such as a nurse, pharmacist, or primary care doctor.

In another embodiment, the individual desiring a diagnostic test enters the user interface portal and requests a test through their individual dashboard. The individual provides demographic information such as age, gender, zip code, and the like, and symptoms. The request is then screened based on clinical guidelines for approval of the test. In some embodiments, the screening is performed by a recommendation system or other machine learning model wherein the algorithm has been trained by multiple data sets and has been approved by regulating authorities. The request for approval is received on the physician dashboard of the system. The request is screened based on clinical guidelines for approval of the test.

In one embodiment of the invention, the input data such as answers to questions, are used as input into specific machine learning models/algorithms such as a recommendation system, classification system, or ensemble algorithm which has been trained on multiple separate data sets. In related embodiments, the set of questions are compliant with FDA regulations for the issuance of a test. Relatedly, approval may also be conducted through a machine learning algorithm developed through codification of current clinical algorithms and further trained on multiple data sets. A medical provider approves delivery of the test kit to the requesting individual and approval is transmitted back through the user interface portal to the clinical testing laboratory. Approvals may be performed in bulk to facilitate mass remote testing

In some embodiments, the user interface device is part of a booth or kiosk, for example an isolated booth or kiosk within a hospital, pharmacy, or convenience store. Providing a device within a pharmacy, hospital, or convenience store with pharmaceutical services allows the individual to receive the test kit from an authorized distributor once notified through the user interface of medical provider approval.

### BRIEF DESCRIPTION OF THE DRAWINGS

FIG. 1 shows steps of a method for individual diagnostic testing.

FIG. 2 shows steps of a method for diagnostic testing with bulk approval of tests.

### DETAILED DESCRIPTION

The invention provides systems and methods for contact-free diagnostic testing. Contact-free means that a person does not need to go see a doctor face-to-face to order a test, receive a test kit, collect and provide a sample, and even receive a test result. Systems and methods of the invention provide a workflow wherein all aspects may be managed using a patient management portal by the clinical testing lab thus streamlining access to diagnostic care. The system manages the interactions between individuals and the testing laboratories, and bridges point-of-care with at-home care. The system provides the clinical testing laboratory a dashboard for receiving, screening, sending for approval, sending and tracking chain-of-custody for kits, receiving returned kits, analyzing and processing the returned kits, and transmitting results to the individual in an anonymized fashion. In some embodiments, the user dashboard includes applications in and translation to various languages to allow for access by non-English-speaking populations. Systems and methods of the invention may use features or assays described in US 2003/0217037 A1; US 2002/0013906 A1; US 2002/0165673 A1; US 2013/0156286 A1; US 2001/0012611 A1; or U.S. Pat. No. 8,380,541 B1, the contents of which are incorporated by reference.

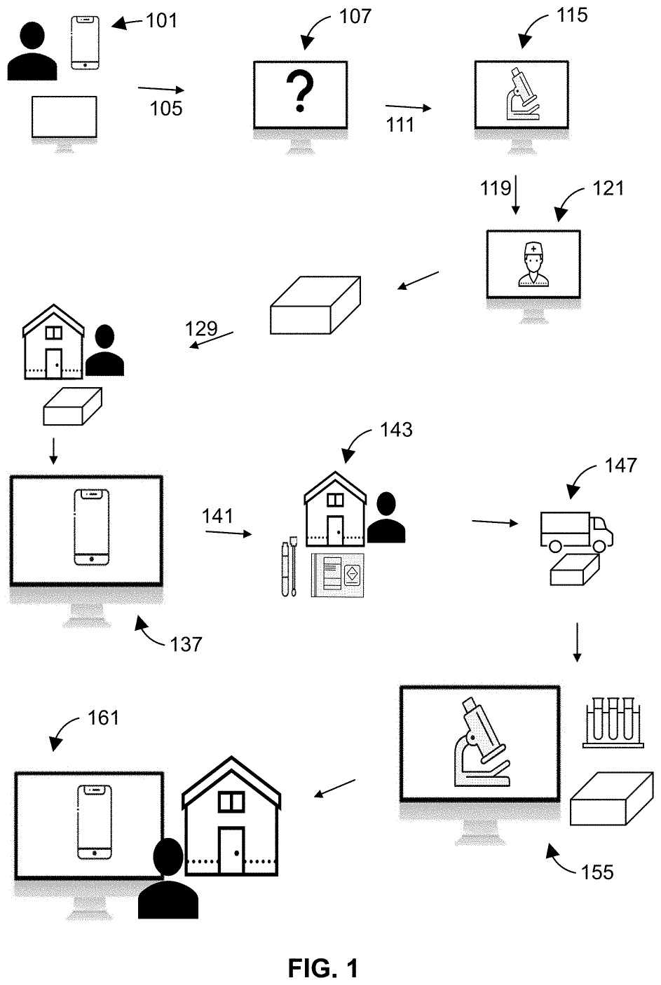

FIG. 1 shows steps of a method for individual testing. An individual signs up 101 for the system on the patient portal e.g., through an app or web-based. Individual requests 105 test(s) based on infectious or communicable disease, or health concern. The system provides 107 questions specific to the test requested and the individual inputs answers to the questions. The system transmits 111 a test request and answers, optionally to a clinical testing laboratory dashboard. The clinical testing laboratory may transmit 119, through the system, the individual's answers to questions and request for a test to an approving medical provider. The medical provider approves 121 and sends approval to clinical testing laboratory. The clinical testing laboratory provides 129 a test kit to individual (e.g., shipping the kit directly or causing the kit to be shipped). In preferred embodiments, the clinical testing laboratory activates 137 the individual's test kit through the system's user dashboard. Optionally, the individual performs a confirmation or counter-activation (e.g., photographs a QR code on the kit). The individual collects 143 a sample, which may be for example, a vaginal swab, urine, excrement, saliva, pus, or finger-stick blood spot at-home. The individual returns 147 sample and test kit back to the clinical testing laboratory. Clinical testing lab processes the kit and analyzes 155 the sample by, for example, performing an assay such as PCR, ELISA, culture, etc. The clinical testing laboratory returns 161 results to the individual via the user dashboard in an anonymized fashion.

The contact-free system for diagnostic testing provides a user interface portal with a dashboard for an individual. The interface may be a computer, a tablet, or smartphone. As depicted, an individual may sign up for the system through the user interface on an app or via a web-based application. The user interface creates a dashboard portal for the individual which allows the person to request a test. Thus, the interaction is initiated by an individual requesting a diagnostic test. When requesting the test, the person may enter demographic information such as age, gender, and zip code into the dashboard display, and answer questions related to the test requested. The individual may request a test based on an infectious or communicable disease, or based on a health concern. The questions may be generated by a machine learning algorithm, or may be authorized by a regulatory agency such as the FDA. The system transmits the individual's request for a test kit and answers to questions to the clinical testing laboratory. The invention provides for a system wherein an individual may request a test kit for any number of tests that can be performed with for example blood, saliva, pus, excrement, urine, or other bodily fluid. Common tests include HbA1c, cholesterol and lipids Test, Fit Colon Cancer Screening, HPV, metabolism, vitamin D and testosterone, vitamin D and inflammation, and sexually transmitted infections. Other examples include tests for iron deficiency or high cholesterol. Tests requested could also be for an enzyme marker such as alkaline phosphatase (ALP), alanine aminotransferase (ALT), aspartate aminotransferase (AST), or bilirubin for risk of cancer or other conditions such as liver cirrhosis, gallbladder inflammation, stroke, hepatitis, or celiac disease. The requested test kit could be for thyroid function panel, kidney function test, or glucose test. An individual may also request various coagulation tests such as a Factor V, Fibrinogen level, prothrombin time, or platelet count. Further the test requested could be for hormone levels indicative of health conditions such as a dehydroepiandrosterone (DHEA) deficiency in men which may indicate type 2 diabetes, kidney disease, or AIDS, where high levels in men or women may indicate cancer or tumor in adrenal glands. Other tests requested may be protein-related tests such as C-reactive protein test (CRP) high levels of which may indicate artery inflammation, inflammatory bowel disease, lupus, rheumatoid arthritis, or heart disease. Other screening tests may include screenings for colorectal cancer or allergic or food sensitivities.

In other embodiments, testing may also be for pathogenic diseases such as HIV, hepatitis C, COVID-19, or for common bacteriological infections such as strep. The self-administered at-home test kit for pathogen testing provides the advantage of avoiding interaction between the person and a medical worker who would otherwise be required to administer the test or collect a sample, preventing an opportunity for the pathogen to spread to the medical worker.

The pathogenic infectious agent to be tested may be a virus, a bacterium, a mycoplasma, a fungus, a yeast, other or micro-organism. The pathogenic agent to be tested may be selected from the group consisting of Influenza A Matrix protein, Influenza H3N2, Influenza H1N1 seasonal, Influenza H1N1 novel, Influenza B, Streptococcus pyogenes (A), Mycobacterium Tuberculosis, Staphylococcus aureus (MR), Staphylococcus aureus (RS), Bordetella pertussis (whooping cough), Streptococcus agalactiae (B), Influenza H5N1, Influenza H7N9, Adenovirus B, Adenovirus C, Adenovirus E, Hepatitis b, Hepatitis c, Hepatitis delta, Treponema pallidum, HSV-1, HSV-2, HIV-1, HIV-2, Dengue 1, Dengue 2, Dengue 3, Dengue 4, Malaria, West Nile Virus, Trypanosoma cruzi (Chagas), Klebsiella pneumoniae (Enterobacteriaceae spp), Klebsiella pneumoniae carbapenemase (KPC), Epstein Barr Virus (mono), Rhinovirus, Parainfluenza virus (1), Parainfluenza virus (2), Parainfluenza virus (3), Parainfluenza virus (4a), Parainfluenza virus (4b), Respiratory syncytial virus (RSV) A, Respiratory syncytial virus (RSV) B, Coronavirus 229E, Coronavirus HKU1, Coronavirus OC43, Coronavirus NL63, Novel Coronavirus, Bocavirus, human metapneumovirus (HMPV), Streptococcus pneumoniae (penic R), Streptococcus pneumoniae (S), Mycoplasma pneumoniae, Chlamydia pneumoniae, Bordetella parpertussis, Haemophilus influenzae (ampic R), Haemophilus influenzae (ampic S), Moraxella catarrhalis, Pseudomonas spp (aeruginosa), Haemophilus parainfluenzae, Enterobacter cloacae (Enterobacteriaceae spp), Enterobacter aerogenes (Enterobacteriaceae spp), Serratia marcescens (Enterobacteriaceae spp), Acinetobacter baumanii, Legionella spp, Escherichia coli, Candida, Chlamydia trachomatis, Human Papilloma Virus, Neisseria gonorrhoeae, plasmodium, and Trichomonas (vagin).

Additionally, the pathogenic agent to be tested may be selected from influenza, a respiratory disease, a sexually transmitted disease, and another infectious disease. The influenza to be tested may be selected from H1N1 (seasonal), H1N1 (novel), H3N2, H7N9, and H5N1. The respiratory disease may be selected from an upper respiratory disease and a lower respiratory disease. Where the disease is a respiratory disease, the disease-causing organism to be tested may be selected from the group consisting of adenovirus B, adenovirus C, adenovirus E, Bordetella pertussis, Mycobacterium tuberculosis (MTB), Staphylococcus aureus, Methicillin-Resistant Staphylococcus aureus (MRSA), Group A streptococcus, Group B streptococcus, Moraxella catarrhalis, Enterobacter aerogenes, Haemophilus parainfluenzae, Metapneumo Virus, Streptococcus pneumonia, Parainfluenza Virus 1, Parainfluenza Virus 2, Parainfluenza Virus 3, Coronavirus OC43, Coronavirus NL63, Coronavirus MERS, Coronavirus HKU1, Coronavirus 229E, Klibsiella pneumonia phoE, Klebsiella pneumonia KPC, Bocavirus type 2,4, and Bocavirus type 1,3.

Where the pathogenic agent is a sexually transmitted disease, the pathogenic disease may be herpes simplex virus (HSV), human immunodeficiency virus (HIV), HIV-2 Group A, HIV-2 Group B, HIV-1 Group M, Hepatitis B, Hepatitis Delta, herpes simplex virus (HSV), streptococcus B, and Treponema pallidum.

Where the pathogenic agent to be tested is an infectious disease, the infectious disease-causing agent selected from the group consisting of West Nile Virus, Epstein-Barr Virus, plasmodium, Trypanosoma cruzi, and a Dengue Virus.

The pathogen to be tested may also be a coronavirus, for example COVID-19. Coronaviruses are enveloped positive-sense RNA viruses, characterized by club-like spikes that project from their surface, for example as described in Fehr and Perlman (2015) “Coronaviruses: an overview of their replication and pathogenesis”, Methods Mol Biol., 1282:1-23, the entirety of the contents of which are incorporated herein by reference.

Once the test is requested, the clinical testing laboratory transmits the individual's request for a diagnostic test and answers to questions to the medical provider. The medical provider approves the request based on to the medical provider for authorization to send the kit to the individual. The request for approval is received on the physician dashboard of the system. The request is screened based on clinical guidelines for approval of the test. In one aspect of the invention, the input data such as answers to questions, are used as input into specific machine learning models/algorithms such as a recommendation system, classification system, or ensemble algorithm which has been trained on multiple separate data sets. The set of questions may be a set of questions compliant with FDA regulations for the test issuance. Approval may also be conducted through a machine learning algorithm developed through codification of current clinical algorithms and further trained on multiple separate data sets.

Authorization receipt from the medical provider within the system triggers the clinical testing laboratory to send the test kit to the individual. The system provides for notifying the individual of the authorization. Providing the test to the individual includes chain-of-custody and tracking protocols, and may be through courier, delivery service, mail, FedEx, UPS, or other means of delivery to an individual. Once received, the clinical testing laboratory, through the system, activates the individual's test kit. Activation includes security protocols such as requiring electronic or biometric identification, bar codes, or two-factor authentication.

The test kit includes instructions for collecting a sample and processing for return. In some embodiments, the test kit will include a pre-paid shipping label and appropriate packaging. The individual may then complete their portion of the test by collecting a sample required by the test, for example a nasal swab, saliva sample, excrement sample, pus, semen, blood or other bodily fluid sample. If the subject is shipping the sample, the packaging for the test may comprise a pre-paid package. The kit contains chain-of-custody tracking and other security features wherein the individual and the clinical testing lab can track the return of the kit to the clinical testing laboratory.

Systems and methods of the invention may also involve bulk approval and testing for multiple individuals.

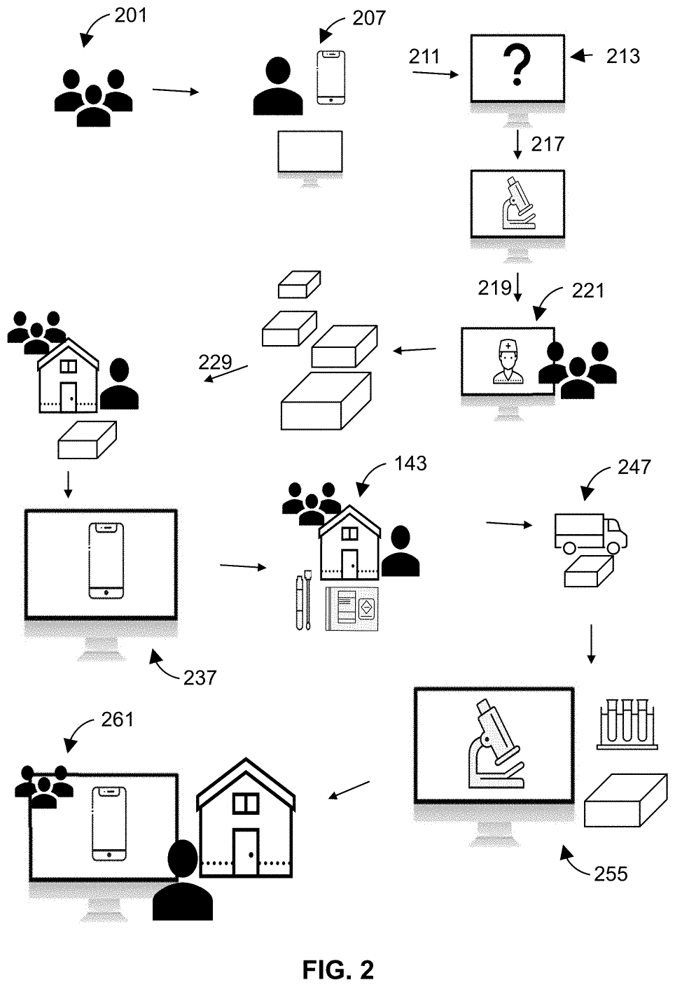

FIG. 2 shows a method that includes bulk approval and testing for multiple individuals. Multiple individuals request 201 tests. Each individual signs 207 up for the system on the user interface dashboard portal either through an app or web-based. Individuals request 211 test(s) based on infectious or communicable disease, or health concern. The system provides 213 questions specific to the test requested; individual inputs answers to questions. The system transmits 217 request and answers to clinical testing laboratory dashboard. The clinical testing laboratory transmits 219, through the system, the multiple individual's answers to questions and request for test kit to approving medical provider. One or more medical providers (e.g., a physician or team) approves 221 (or authorizes or “orders”) the tests en masse and sends approval to clinical testing laboratory. The clinical testing laboratory provides test kits to individuals. In preferred embodiments, the clinical testing laboratory activates 237 the individuals' test kit through the system's user dashboard. Each individual collects 243 a sample such as a vaginal swab, urine, excrement, saliva, pus, or finger-stick blood spot at-home. The individuals return 247 the samples and test kits back to clinical testing laboratory. Once returned to the clinical testing laboratory, the kit is analyzed and processed. Results are returned to the individual through the system via the user dashboard portal. Clinical testing lab processes 255 the kit and analyzes the sample. Clinical testing laboratory returns 261 results to the individual via the user dashboard in an anonymized fashion.

Whether being used for individual diagnostic testing or for diagnostic testing with bulk approval of tests, methods of the invention include using the system to obtain, from at least one person, a request for a medical test and answers to questions selected based on the medical test. The methods include sharing the request and answers with a medical provider. The medical provider approves 121 and sends approval to clinical testing laboratory. The methods include recording from the medical provider the approval as an order for the medical test. The methods include authorizing a laboratory to provide 129 a medical test kit to the person. The clinical testing laboratory provides 129 a test kit to individual (e.g., shipping the kit directly or causing the kit to be shipped). After at least one individual returns 147 a sample to the laboratory, the laboratory processes the kit and analyzes 155 the sample.

Any suitable analysis may be performed. Preferably the analysis includes at least one clinical diagnostic test. For example, the test may include an assay for a molecular marker or indicia of a disease or condition. In some embodiments, the molecular marker is a molecule, such as O2 or CO2. In some embodiments, the molecular marker is a biomolecule such as a nucleic acid, protein, or sugar. The assay may be any suitable assay such as an immunoassay for a protein (e.g., a free protein or a membrane-bound, e.g., cell-surface protein). The assay may be nucleic acid detection assay such as a “gene chip” or an amplification-based assay such as polymerase chain reaction (PCR) or digital PCR. The assay may include sequencing nucleic acid such as by nucleic acid extraction from a blood or tissue sample, library prep, and sequencing e.g., on next-generation sequencing (NGS) instrument such as an Illumina HISEQ instrument.

In preferred embodiments, the assay is a simple and rapid test such as a PCR test to detect genetic sequences of an infectious agent such as a virus or bacteria. Primers and sequences for detecting infectious agents such as COVID or a sexually transmitted infection (STI) such as chlamydia or gonorrhea are known and may be used for the rapid detection of such infections.

Whether individual test embodiments, or en masse test authorization embodiments, methods may include accepting, by a system of the invention, input of a test result from the laboratory and providing access to the test result to the individual of that test.

As discussed, the system may be used for mass testing such as for a university, business, or suspected outbreak of infectious disease. In this way, the invention provides for mass remote diagnostic testing. The clinical testing laboratory may receive identical or related requests from multiple individuals for diagnostic testing. For example, if a university requires students to be tested for COVID-19 before they are allowed on campus, the university may have students sign up through the system with their own user dashboard and request a COVID-19 test. As above, the student will enter demographic data and answer questions related to the test kit requested. The requests are received from individuals via the clinical testing laboratory dashboard and are presented to clinicians via the medical provider's dashboard for approval en masse. Relatedly, the medical provider's dashboard is code-compliant for mass approvals. Thus, the invention provides for mass approval by the medical provider to facilitate mass remote diagnostic testing.

In the embodiment, multiple individuals may sign up for the system and create individual user dashboards, request tests, and answer questions. The mass requests and inputs are received by the clinical testing laboratory and sent through the system to the medical provider dashboard for approval en masse. Once mass approval is received, the clinical testing laboratory delivers the test kits to the mass individuals. Delivery may be through courier, FedEx, UPS, or some other means for mass delivery. The delivery may take place en masse in a secure location or may be delivered directly to the requesting individual. The system provides security and chain-of-custody features such as those described above. The clinical testing laboratory activates the test kit for individuals as described above. The individual performs their portion of the test kit by collecting a sample such as a vaginal swab, urine, semen, nasal swab, saliva, pus, a finger-stick blood spot, or other bodily fluid as required by the test. Because collection of these samples may be sensitive to subjects, the present invention provides the advantage of allowing the subject to collect the sample themselves in their own home.

The test kit is returned to the clinical testing laboratory as described above and analyzed and processed. Results are returned to each individual through the system via the user dashboard portal in an anonymized fashion. Results are reported on an individual basis to each requesting individual through their specific user dashboard. Once results of the diagnostic test is communicated to the individual, the individual may then follow up with a medical care provider for follow-up care as necessary. the hospital, pharmacy, or convenience store for medical treatment.

In one embodiment of the invention, the input data such as answers to questions, are used as input into specific machine learning models/algorithms such as a recommendation system, classification system, or ensemble algorithm which has been trained on multiple separate data sets. In related embodiments, the set of questions are compliant with FDA regulations for the issuance of a test. Relatedly, approval may also be conducted through a machine learning algorithm developed through codification of current clinical algorithms and further trained on multiple data sets. A medical provider approves delivery of the test kit to the requesting individual and approval is transmitted back through the user interface portal to the clinical testing laboratory.

As discussed above, methods of the invention include using a computer system to perform any combination of the following steps in any suitable order: obtaining a request from a person for a medical test and answers to questions selected based on the medical test; sharing the request and answers with a medical provider; recording in the computer system an order from the medical provider for the medical test; authorizing a laboratory to ship a medical test kit to the person; accepting input of a test result from the laboratory; and providing the person with access to the test result. Interestingly, the methods are effective even if steps are performed not in the recited order. For example, clinical results may be provided most rapidly by authorizing shipment of the medical test kit before (or independently of) when the medical provider authorizes (or “orders”) the test. Activation of the test by the laboratory (and optional counter-activation by the user) may be used to validate that the shipped test kit corresponds to a medical test that was “ordered” by a physician. The computer system manages control and information flow of all such steps. Methods according to certain embodiments are performed by a computer system that includes at least one processor coupled to non-transitory memory containing instructions useful to cause the computer system to perform the method and at least one input/output (I/O) device. Systems of the invention include one or more computer devices that include one or more processors (e.g., a central processing unit (CPU), a graphics processing unit (GPU), etc.), computer-readable storage devices (e.g., main memory, static memory, etc.), or combinations thereof which communicate with each other via a bus.

A processor may be any suitable processor known in the art, such as the processor sold under the trademark XEON E7 by Intel (Santa Clara, Calif.) or the processor sold under the trademark OPTERON 6200 by AMD (Sunnyvale, Calif.). Memory preferably includes at least one tangible, non-transitory medium capable of storing: one or more sets of instructions executable to cause the system to perform functions described herein (e.g., software embodying any methodology or function found herein); data (e.g., embodying any tangible physical objects such as the genetic sequences found in an infectious agent); or both. Systems of the invention may be embodied in or use server- or could-based systems such as, for example, Amazon Web Services. In some embodiments, storage is provided by Amazon Elastic Block Store (Amazon EBS) snapshots, allowing a cloud resource to dynamically mount Amazon EBS volumes with the data needed to perform the methods. Using such resources, the computer system is operable to: provide a patient portal to each of a plurality of test subjects, each via a display in a browser or app; provide a physician portal to at least one medical provider; operate, for the laboratory, a laboratory dashboard allowing laboratory personnel to manage test requests and results; and coordinate all the described steps of the methods. Input/output devices according to the invention may include a video display unit (e.g., a liquid crystal display (LCD) monitor), an alphanumeric input device (e.g., a keyboard), a cursor control device (e.g., a mouse or trackpad), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem. Preferably, an of the patient portal, physician portal, and laboratory dashboard are provided at least in part by the system. In some embodiments, affiliated cloud computing resources contributes the functionality of one or more of those modules. Computer program instructions can be written using any suitable language known in the art including, for example, Perl, BioPerl, Python, C++, C#, JavaScript, Ruby on Rails, Groovy and Grails, or others. Program code can be linear, object-oriented, or a combination thereof. Preferably, program instructions for the tools described here are provided as distinct modules, each with a defined functionality. Exemplary languages, systems, and development environments include Perl, C++, Python, Ruby on Rails, JAVA, Groovy, Grails, Visual Basic .NET. Systems of the invention can be developed using the Ruby programming language and optionally BioRuby, Ruby on Rails, or a combination thereof. Ruby or BioRuby can be implemented in Linux, Mac OS X, and Windows as well as, with JRuby, on the Java Virtual Machine, and supports object oriented development. See Metz, Practical Object-Oriented Design in Ruby: An Agile Primer, Addison-Wesley (2012) and Goto, et al., BioRuby: bioinformatics software for the Ruby programming language, Bioinformatics 26(20):2617-2619 (2010).

Systems and methods of the invention can be developed using the Groovy programming language and the web development framework Grails. Grails is an open source model-view-controller (MVC) web framework and development platform that provides domain classes that carry application data for display by the view. Grails domain classes can generate the underlying database schema. Grails provides a development platform for applications including web applications, as well as a database and an object relational mapping framework called Grails Object Relational Mapping (GORM). The GORM can map objects to relational databases and represent relationships between those objects. GORM relies on the Hibernate object-relational persistence framework to map complex domain classes to relational database tables. Grails further includes the Jetty web container and server and a web page layout framework (SiteMesh) to create web components. Groovy and Grails are discussed in Judd, et al., Beginning Groovy and Grails, Apress, Berkeley, Calif., 414 p. (2008); Brown, The Definitive Guide to Grails, Apress, Berkeley, Calif., 618 p. (2009).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A testing method comprising:
obtaining, by a computer system, a request from a person for a medical test and answers to questions selected based on the medical test;
sharing the request and answers with a medical provider;
recording in the computer system an order from the medical provider for the medical test;
authorizing a laboratory to ship a medical test kit to the person;
accepting input of a test result from the laboratory; and
providing the person with access to the test result.

2. The method of claim 1, wherein the obtaining step includes having the person: sign-in to access the system, make the request for the medical test, and give the answers to the questions selected based on the medical test.

3. The method of claim 1, wherein the system presents the person with the questions in response to receiving the request for the medical test from the person.

4. The method of claim 1, wherein the authorizing step includes informing the laboratory that the medical provider ordered the test and notifying the person that the test kit is activated.

5. The method of claim 1, wherein the computer system includes at least one processor coupled to non-transitory memory containing instructions useful to cause the computer system to perform the method.

6. The method of claim 1, wherein the computer system is operable to:
provide a patient portal to each of a plurality of test subjects, each via a display in a browser or app;
provide a physician portal to at least one medical provider; and
operate, for the laboratory, a laboratory dashboard allowing laboratory personnel to manage test requests and results.

7. The method of claim 6, wherein the person is shown the questions selected based on the medical test through his or her respective patient portal.

8. The method of claim 1, wherein the computer system provides functionality allowing the laboratory to activate the medical test kit.

9. The method of claim 8, wherein the computer system via a patient portal confirms receipt by the person of the activated medical test kit.

10. The method of claim 1, wherein the received request is for a medical test for an infection, wherein the order requires the medical test kit to include a collection device for a biological sample, and wherein the test result input by the laboratory is obtained by performing a laboratory assay on the biological sample.

11. The method of claim 10, wherein the infection is a sexually transmitted infection, the collection device includes a genital swab, and the laboratory assay includes a test for the presence of one or more molecular markers of chlamydia and/or gonorrhea.

12. The method of claim 10, wherein the infection is a respiratory virus, the collection device includes a nasal swab, and the laboratory assay includes a test for the presence of one or more molecular markers of the respiratory infection.

13. The method of claim 1, wherein the computer system receives requests from a plurality of people for the same test and further wherein the computer system:
aggregates test requests based on clinical similarities in a plurality of answers from the respective people;
shares a single aggregate request with the medical provider; and
records from the medical provider a bulk authorization of orders for medical tests for the plurality of people.

14. The method of claim 1, wherein the computer system is operable to:
operate, for the laboratory, a laboratory dashboard allowing laboratory personnel to manage test requests and results; and
read from, and update, records in a laboratory information management system of the laboratory.

15. The method of claim 1, wherein the computer system is operable to:
provide a physician portal to at least one medical provider; and
authorize the laboratory to ship the medical test after, and only after, recording from the medical provider the order for the medical test.

16. The method of claim 15, wherein the step of providing the person with access to the test result is performed before the person has met the medical provider face-to-face.

17. The method of claim 16, wherein the test result includes the presence or absence of a sexually-transmitted infection.

18. The method of claim 16, wherein the computer system aggregates test requests from a plurality of people into a single aggregate request to the medical provider; and
authorizes, upon receiving an approval of the aggregate request from the medical provider, the laboratory to ship test kits to each of the plurality of people.

19. The method of claim 18, wherein computer system operates a laboratory dashboard to receive and manage results from the test kits, wherein the results include test results for an epidemic or pandemic disease or condition.

20. The method of claim 1, wherein the computer system includes a machine learning system that operates on the answers to make a patient classification or test recommendation.

21. The method of claim 20, wherein the patient classification or the test recommendation is provided to the laboratory or the medical provider.

22. The method of claim 1, wherein the computer system is operable to:
provide a physician portal to the medical provider;
screen the request based on clinical guidelines for the medical test; and
present the screened request to the medical provider via the physician portal.

23. The method of claim 1, wherein authorization for the requested test triggers distribution of the test kit to the requesting individual.

24. The method of claim 1, further comprising providing the medical test kit to the person by sending to the medical test kit to: an address for the person, or a participating pharmacy or clinical site.

25. The method of claim 1, wherein the authorizing step includes informing the laboratory that the medical provider ordered the test and prompting the person to activate the test.

26. The method of claim 1, wherein the computer system documents a chain of custody for the medical test kit and results.
